(19) United States
(12) Patent Application Publication
(10) Pub. No.: US 2021/0001820 A1
(43) Pub. Date: Jan. 7, 2021

(54) VEHICLE OCCUPANT PROTECTION

(71) Applicant: International Truck Intellectual Property Company, LLC, Lisle, IL (US)

(72) Inventor: Tom Lee Sorensen, Naperville, IL (US)

(73) Assignee: International Truck Intellectual Property Company, LLC, Lisle, IL (US)

(21) Appl. No.: 17/026,496

(22) Filed: Sep. 21, 2020

(51) Int. Cl. B60S 1/64; A61L 9/20

(52) U.S. Cl. CPC B60S 1/64; A61L 9/20; A61L 2209/16; A61L 2209/12; A61L 2209/11

(57) ABSTRACT

Embodiments disclosed herein relate to a system and a method for sterilizing air in a vehicle. In one embodiment, the system comprises an HVAC system comprising a duct disposed in the vehicle. At least one source of electromagnetic radiation is located in the duct. A controller is disposed on the vehicle and is connected with the at least one source of electromagnetic radiation to determine operation of the at least one source of electromagnetic radiation.

### BACKGROUND

This disclosure relates generally to a system and a method of protecting an occupant of a vehicle. More specifically, this disclosure relates to a system and a method protecting an occupant of a school bus, a commercial vehicle and the like, from an airborne contaminant.

Currently, many vehicles are ventilated mechanically with an HVAC system that circulates air within a cabin of the vehicle. This circulated air is shared among occupants, such as a driver and passengers of a school bus, multiple drivers of a commercial vehicle, and the like of the vehicle. Any of these occupants can introduce a contaminant to circulated air thereby contaminating the HVAC system, a vehicle passenger cabin, and a vehicle driver cabin. Furthermore, elements of a vehicle, such as an air conditioner coil and the like, may present an attractive environment for a biological contaminant, such as a microorganism, viz. bacteria, viruses, fungi and parasites. Some contaminants may be incorporated into the circulated air. Anything incorporated into the circulated air can impact an occupant of the vehicle. Additionally, a contaminant may be present in air outside of the vehicle. This contaminant can be introduced into the vehicle, e.g. when a door or a window on the vehicle is opened. Accordingly, it is desirable to have a system and a method that protects an occupant of a vehicle from an airborne contaminant.

### SUMMARY

Embodiments disclosed herein relate to a system and a method for sterilizing air in a vehicle. In one embodiment, the system comprises an HVAC system comprising a duct disposed in the vehicle. At least one source of electromagnetic radiation is located in the duct. A controller is disposed on the vehicle and is connected with the at least one source of electromagnetic radiation to determine operation of the at least one source of electromagnetic radiation.

### DESCRIPTION OF THE DRAWINGS

FIG. 1 is a generic schematic diagram of a vehicle HVAC system including an embodiment of a system for protecting an occupant of a vehicle disclosed herein; and

FIG. 2 is a generic view of a vehicle including the system of FIG. 1.

### DETAILED DESCRIPTION

This disclosure relates specifically to a system and a method for protecting an occupant of a vehicle, such as a school bus, a commercial vehicle and the like. Embodiments disclosed herein generally comprise an HVAC system 10 comprising a duct 11 disposed in a vehicle 13. The system for sterilizing air in the vehicle 13 includes at least one source 12 of electromagnetic radiation located in the duct 11 of the HVAC system 10. Other elements of the HVAC system 10 include, but are not limited to, duct 18 that conveys air to a vent in the vehicle 13, a duct 20 that conveys air to a vent associated with a defroster, an operable door 22 that controls air movement to the duct 20, a duct 24 that conveys air to a vent on the vehicle 13, an operable door 26 that controls air movement to the duct 24, a heater core 28 that imparts thermal energy to air, an operable door 30 that controls air movement from the heater core 28, an evaporator core 32 that absorbs thermal energy from air, a blower 34 that moves air, an operable door 36 that controls air movement into a cabin of the vehicle 13, an air filter 38, a duct 40 that conveys air from an exterior 19 of the vehicle 13, and a duct 36 that conveys air to a cabin of the vehicle 13.

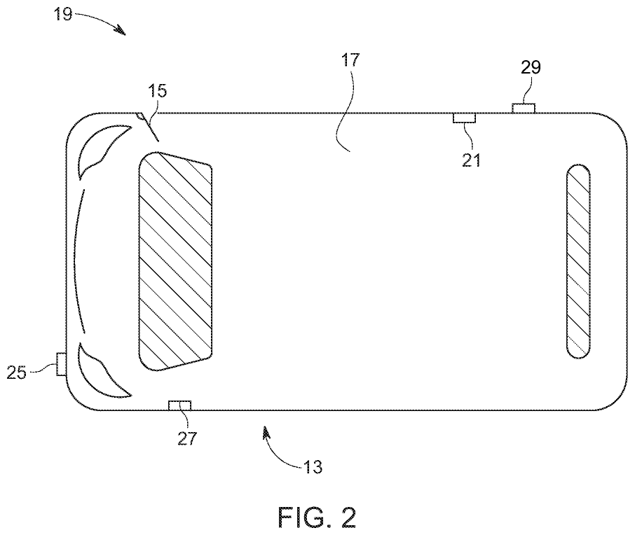

As shown in FIG. 2, the vehicle 13 includes at least one portal 15, such as a door, a window and the like, through which air passes between an interior 17 of the vehicle 13 and an exterior 19 of the vehicle 13. At least one sensor 21 is disposed on the interior 15 of the vehicle 13, and at least one sensor 25 is disposed on the exterior 19 of the vehicle 13. Both of the at least one sensors 21 and 25 sample air adjacent the at least one sensors 21 and 25 to detect quality of that air including, but not limited to, presence of an airborne contaminant, such as carbon monoxide, carbon dioxide, diesel particulate, pollen, viruses, bacteria, vapors, aerosols and the like. The vehicle 13 includes at least one pressure sensor 27 disposed on the interior 17 of the vehicle 13 and at least one pressure sensor 29 disposed on the exterior 19 of the vehicle 13. The at least one sensors 21 and 25 and the at least one pressure sensors 27 and 29 are connected, wired, wirelessly or otherwise, to a controller 14, which can comprise one or more data processors, on the vehicle 13. In some embodiments, the controller 14 is augmented by at least one data processor located outside of the vehicle 13 and connected with the vehicle 13 by appropriate means, such as wireless connection to the internet and the like. In some embodiments, the at least one sensors 21 and 27 disposed on the interior 17 of the vehicle 13 may comprise a single sensor. Also, in some embodiments, the at least one sensors 25 and 29 disposed on the exterior 19 of the vehicle 13 may comprise a single sensor.

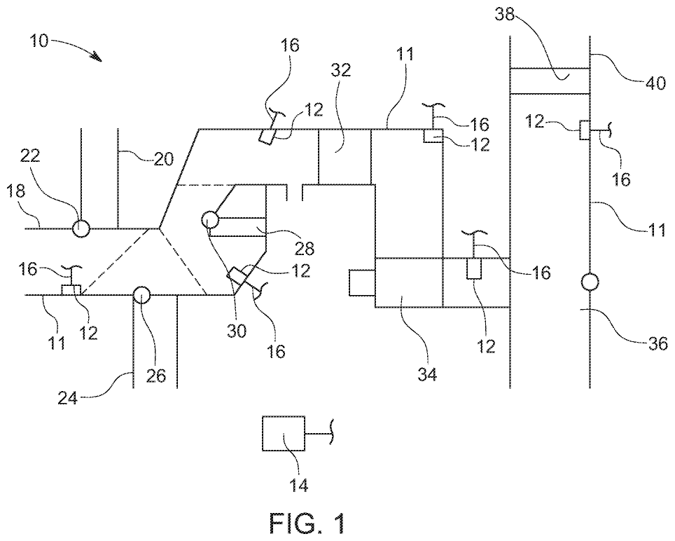

Returning to FIG. 1, in some embodiments, the at least one source 12 comprises at least one of a light emitting diode, low power light emitting diode, a mercury vapor light and the like. There are six (6) at least one sources 12 in the embodiment shown in FIG. 1. In the illustrated embodiment, at least one source 12 is disposed on the duct 18 that convey air to the vehicle vent, at least one source 12 is disposed adjacent the duct 20 that conveys air to a vent associated with a defroster, at least one source 12 disposed adjacent the heater core 28 , at least one source 12 is disposed adjacent the evaporator core 32, at least one source 12 is disposed adjacent the blower 34, and at least one source 12 adjacent the air filter 38.

The electromagnetic radiation has a wavelength within the range of about 200 nanometers to about 280 nanometers, with 260 nanometers being an absorbance wavelength of some nucleic acids. The at least one sources 12 are connected with the controller 14 by a conductor 16, either wired or wirelessly. The controller 14 is disposed on the vehicle. In some embodiments, the controller 14 comprises a body controller of a vehicle. The at least one sources 12 are positioned within the HVAC system 10 at locations empirically determined to sterilize air circulating within the HVAC system 10 and to sterilize portions of the HVAC system 10 illuminated by the electromagnetic radiation from the at least one sources 12. Locations of the at least one sources 12 are chosen to reduce likelihood of unintended exposure to the electromagnetic radiation from the at least one sources 12, i.e. reduce likelihood of exposure of the vehicle occupant to the electromagnetic radiation.

The controller 14 determines operation of the at least one sources 12, viz. time when the at least one sources 12 emit electromagnetic radiation. The controller 14 can determine intensity of the electromagnetic radiation. The controller 14 can be programmable. The controller 14 can allow the at least one sources 12 to emit electromagnetic radiation, for instance, at specific times of day, for specific temporal duration, etc. The controller 14 may allow the at least one sources 12 to emit electromagnetic radiation at specific times to manage vehicle power usage. In some embodiments, the controller 14 may allow the at least one sources 12 to emit electromagnetic radiation substantially continuously thereby enabling substantially continuous sterilization of the HVAC system 10.

With structure of a system for sterilizing air in a vehicle being described, now attention is drawn to a method of sterilizing air in a vehicle.

Once the vehicle 13 includes an HVAC system 10 with at least one source 12 of electromagnetic radiation having a wavelength within a range of about 200 nanometers to about 280 nanometers located in a duct 11 of the HVAC system 10, then temporal duration of emission of the electromagnetic radiation from the at least one source 12 of electromagnetic radiation and intensity of electromagnetic radiation are determined. These parameters are inputted into the controller 14. The controller 14 energizes the at least one source 12 of electromagnetic radiation in accordance with the inputted parameters.

The at least one sensor 25 samples air outside of the vehicle 13 periodically for air quality and reports results to the controller 14. Similarly, the at least one sensor 21 on the interior 17 of the vehicle 13 periodically samples quality of the air on the interior 17 of the vehicle 13 and reports results to the controller 14. The controller 14 compares results from the at least one sensor 21 and the at least one sensor 25, thereby determining if air inside the vehicle 13 or air outside the vehicle 13 has less contaminants.

The at least one pressure sensor 27 on the interior 17 of the vehicle 13 and the at least one pressure sensor 29 on exterior 19 of the vehicle 13 read the air pressure on both the interior 17 and exterior 19 of the vehicle 13, respectively. Results from those at least one pressure sensors 27 and 29 are sent to the controller 14. The controller 14 compares results from the at least one pressure sensor 27 and the at least one pressure sensor 29 thereby determining a pressure differential between the interior 17 of the vehicle 13 and the exterior 19 of the vehicle 13. This pressure differential can influence air flow through the portal 15 when the portal 15 is opened.

If air on the exterior 19 of the vehicle 13 contains less contaminants than air on the interior 17 of the vehicle 13, the HVAC system 10 can regulate air flow between the exterior 19 of the vehicle and the interior 17 of the vehicle 13. To provide air a reduced amount of contaminants, the HVAC system 10 can operate on a substantially continuous basis to sterilize the air of the vehicle 13. The controller 14, using results from the at least one pressure sensor 27, the at least one pressure sensor 29, the at least one sensor 25 and the at least one sensor 21, regulates air pressure in the vehicle 13 to create a pressure differential favoring movement of air with reduced amount of contaminants into or out of the vehicle 13.

For instance, to prepare for an onboarding passenger to enter the vehicle 13 through the portal 15, the controller 14 uses the above-discussed results to determine if air on the interior 17 of the vehicle 13 or air on the exterior 19 of the vehicle 13 has lower contaminants. If air on the interior 17 of the vehicle 13 has lower contaminants than air on the exterior 19 of the vehicle 13, the HVAC system 10 adjusts air pressure on the interior 17 of the vehicle 13 such that when the portal 15 opens, air from the interior 17 of the vehicle 13 will move towards the exterior 19 of the vehicle 13. If air on the exterior 19 of the vehicle 13 has lower contaminants than air on the interior 17 of the vehicle, the HVAC system 10 will create reduced air pressure on the interior 17 of the vehicle 13 with respect to air pressure on the exterior 19 of the vehicle 13, thereby drawing air from the exterior 19 of the vehicle 13 to the interior 17 of the vehicle 13 through the portal 15. In either case, the air containing a higher level of contaminants moves away from the interior 17 and an occupant of the vehicle 13.

What is claimed is:

1. A system for sterilizing air in a vehicle, the system comprising:
an HVAC system comprising a duct disposed in the vehicle;
at least one source of electromagnetic radiation located in the duct; and
a controller disposed on the vehicle and connected with the at least one source of electromagnetic radiation to determine operation of the at least one source of electromagnetic radiation.

2. The system as defined in claim 1 wherein the at least one source of electromagnetic radiation comprises a light emitting diode.

3. The system as defined in claim 1 wherein the electromagnetic radiation has a wavelength within a range of 200 nanometers to 280 nanometers.

4. The system as defined in claim 1 wherein there are six at least one source of electromagnetic radiation located in the duct.

5. The system as defined in claim 1 wherein the at least one source of electromagnetic radiation comprises a mercury vapor light.

6. The system as defined in claim 1 further comprising:
at least one of a defroster, a heater core, an evaporator core, a blower and an air filter located in the HVAC system; and
at least one of the at least one source of electromagnetic radiation being disposed adjacent the at least one of a defroster, a heater core, an evaporator core, a blower and an air filter.
